SEQUENCE LISTING (2 sequences)

SEQ ID NO: 1 - DNA, 20 bp, Artificial Sequence
  Description of Artificial Sequence PCR amplification primer
ccacgacggtgacacgcttc

SEQ ID NO: 2 - DNA, 19 bp, Artificial Sequence
  Description of Artificial Sequence PCR amplification primer
caagcaagggttcaaagac

(19) United States
(12) Patent Application Publication
(10) Pub. No.: US 2005/0281785 A1
(43) Pub. Date: Dec. 22, 2005

(54) Method of reducing side effects of cancer therapy using p53 recombinant adenovirus

(76) Inventors: Zhaohui Peng, Shenzhen (CN); Xiaozhi Zhang, Shenzhen (CN)

Correspondence Address: BEN WANG, 706 COLORADO AVE, PALO ALTO, CA, 94303, US

(21) Appl. No.: 11/075,035

(22) Filed: Mar. 7, 2005

Related U.S. Application Data
(63) Continuation-in-part of application No. PCT/CN04/00465, filed on Mar. 8, 2004.

(30) Foreign Application Priority Data
Jan. 26, 2005 (CN) 200510002779.1

(51) Int. Cl. A61K 48/00

(52) U.S. Cl. 424/93.2

(57) ABSTRACT

The present invention discloses that recombinant p53 adenovirus can ameliorate the side effects normally associated with chemotherapy and radiotherapy. Administration of recombinant p53 adenovirus is effective in reducing the side effects of cancer therapy on patients, and improving the biochemical parameters of these patients. The invention also discloses that recombinant p53 adenovirus alone can improve the quality of life for cancer patients without being combined with other forms of cancer therapy such as chemotherapy or radiotherapy.

### RELATED APPLICATION

This application is a continuation in part of International Application PCT/CN2004/00465, filed Mar. 8, 2004. This application also claims priority to Chinese Patent Application No.: 200510002779.1, filed Jan. 26, 2005. The entire content of both applications mentioned above are incorporated herein by reference.

### FIELD OF THE INVENTION

The present invention relates to methods of improving the quality of life of individuals suffering from cancer and reducing the side effects caused by cancer therapy such as chemotherapy, radiotherapy and other types of cancer therapy that these individuals go through.

### BACKGROUND OF THE INVENTION

Cancers are a leading cause of death in animals and humans. Common forms of cancer therapy today include surgery, radiotherapy and chemotherapy. In spite of advances in the field of cancer treatment, each of these known therapies has serious side effects. For example, surgery disfigures the patient or interferes with normal bodily functions. Chemotherapy or radiotherapy may cause patients develop acute debilitating symptoms including nausea, vomiting, diarrhea, hypersensitivity to light, hair loss, etc. The side effects of these cancer therapies frequently limit the frequency and dosage at which they can be administered. Indications of these side effects can be demonstrated through biochemical and physiological tests such as WBC, HB, AST, and ALT. These tests tend to show that chemotherapy and radiation therapy cause treated patients to have below-normal measurements as compared to those control individuals without cancer treatment. Often times, cancer patients suffer greatly from these side effects when they are going through chemotherapy or radiation therapy. In some cases, to the patient, the pain and suffering derived from these side effects are even greater than that of the cancer itself.

Attempts have been made to reduce the serious side effects of these cancer therapies.

U.S. Pat. No. 6,479,500 describes the structure and function of chemical agents that can reduce the side effects caused by anti-cancer agent.

U.S. Pat. No. 5,017,371 describes the use of interferon in reducing the toxic side effects associated with radiation therapy or chemotherapy.

U.S. Pat. No. 6,462,017 describes methods of reducing the severity of chemotherapy side in cancer patients using thymosin alpha in combination with chemotherapy.

Despite of the various attempts mentioned above, patients all over the world still suffers from the ills of cancer therapy, and are constantly searching for ways to improve their quality of life. Thus, it is clear that there exists a great need to invent effective and novel means to reduce side effects of cancer therapies in order to improve the quality of life for cancer patients going through either chemotherapy or radiation therapy.

### SUMMARY OF THE INVENTION

In accordance with one aspect of the present invention, a method is provided in which the side effects of chemotherapy or radiation therapy in cancer patients are reduced by administering a recombinant p53 adenovirus in an amount that is effective to reduce side effects, in conjunction with the administration of the chemotherapy agent to the patient. The reduction in the severity of post-chemotherapy side effects increases the quality of life experienced by patients receiving chemotherapy or radiotherapy. These improvements are shown in the confirmatory testing of biological, physiological and biochemical standards.

According to another aspect of the present invention, the composition containing recombinant p53 adenovirus can be administered before the administration of cancer therapy such as chemotherapy or radiotherapy.

According to yet another aspect of the present invention, the composition containing recombinant p53 adenovirus can be administered after the administration of cancer therapy such as chemotherapy or radiotherapy.

In accordance with another aspect of the present invention, a method is provided in which the quality of life of cancer patients is improved via the administration of a recombinant p53 adenovirus. As an indication of such improved quality of life, the cancer patient is observed to have better appetite, better sleep, higher level of energy, less pain, and desired weight gain.

### BRIEF DESCRIPTION OF THE FIGURES

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1A and FIG. 1B show the schematic process of the construction of a recombinant p53 adenovirus.

FIG. 2 shows the flow chart of the experimental protocols for the production of the recombinant p53 adenovirus.

FIG. 3 shows the result of an agarose gel electrophoresis of a PCR amplification of the recombinant p53 adenovirus after generations of passage, using p53 cDNA as template, and 5′CCACGACGGTGACACGCTTC3′, and 5′CAAGCAAGGGTTCAAAGAC3′ as primers. This result confirms the stability of the recombinant p53 adenovirus. A DNA fragment of 1400 bp was obtained after PCR amplification. Lane 1: DNA molecular weight standard markers; Lanes 2, 3, 4: Results of the PCR amplification of p53 cDNA.

FIG. 4 shows the Western Blot results of the recombinant p53 adenovirus, purified from lysed cells transfected with the recombinant p53 adenovirus 36 hours earlier. The transfected cells were human laryngeal cancer cells Hep-2, and non-small cell cancer cells H1299. Lane 1: Protein molecular weight standard markers; Lanes 2 and 3: Negative controls showing Western Blot results from Hep-2 cells and H1299 cells before they were transfected with recombinant p53 adenovirus (Ad-p53), respectively; Lanes 4 and 5: Western Blot results from Hep-2 cells and H1299 cells after they were transfected with recombinant p53 adenovirus (Ad-p53), respectively.

FIG. 5 shows a graph demonstrating recombinant p53 adenovirus's killing effect on Hep-2 cells. Hep-2 cells were cultured on six-well plates until their density reaches 1×10⁶ per well. Ad-p53 was administered to transfect the cells at 100 MOI at different time intervals (24, 48, 72, and 96 hours). The killed cells are represented in percentage by the color blue as stained with Trypan Blue.

FIG. 6 shows a flow chart of applying recombinant p53 adenovirus in clinical studies of its effect on improving patient's quality of life, welfare and reducing the side effects of cancer therapy. The chart includes procedures of clinical observation and methods of evaluating the positive effects of the recombinant p53 adenovirus.

### DETAILED DESCRIPTION OF THE INVENTION

The present invention arose during the course of clinical trials using a recombinant p53 adenovirus to cure various forms of cancers such as head and neck squamous cancer, non-small cell cancer, liver cancer, lung cancer, thyroid cancer, cervical cancer, etc. Independent of its abilities in reducing cancer growth, it was discovered that recombinant p53 adenovirus also possesses a separate function of reducing the side effects of conventional chemotherapy and radiation therapy. In addition, the recombinant p53 adenovirus can also by itself improve the quality of life of cancer-suffering patients. Below, we first describe the process of constructing and producing the recombinant p53 adenovirus. We then describe the use of the adenovirus in treating individuals with cancer. A preferred embodiment of the recombinant p53 adenovirus is named Gendicine, the structure of which is described in Chinese Patent No.: ZL 02115228.4, and the content of the Chinese patent is incorporated herein in its entirety.

As used herein, the term “subject” encompasses an animal, such as a mammal, and preferably a human.

As used herein, the term “cancer therapy” refers to chemotherapy, radiotherapy, and other common therapeutic methods used in modern medicine.

As used herein, the term “side effect” or “side effects” refers to the negative symptoms such as nausea, headache, loss of sleep, loss of appetite, and the like, caused by a cancer therapy, as compared to those who have not been treated with the cancer therapy.

As used herein, the term “transfection” is used to describe the targeted delivery of DNA to eukaryotic cells using delivery systems, such as, adenoviral, AAV, retroviral, or plasmid delivery gene transfer methods. The specificity of viral gene delivery may be selected to preferentially direct the gene to a particular target cell, such as by using viruses that are able to infect particular cell types.

As used herein, the term “an amount effective” refers to an amount of the recombinant p53 adenovirus which is sufficient to alleviate the side effects of a cancer therapy on a subject suffering from cancer.

As used herein, the term “quality of life” generally refers to the well being of a cancer patient, including both physical and mental aspects of the patient's life.

1. p53 and p53 Mutations in Cancer

P53 is currently recognized as a tumor suppressor gene (Montenarh, M., “Biochemical, Immunological, and Functional Aspects of the Growth-Suppressor/Oncoprotein p53,” Critical Reviews in Oncogenesis, (3): 233-256, 1992). High levels have been found in many cells transformed by chemical carcinogenesis, ultraviolet radiation, and several viruses, including SV40. The p53 gene is a frequent target of mutational inactivation in a wide variety of human tumors and is already documented to be the most frequently-mutated gene in common human cancers (Mercer, W. Edward, “Cell Cycle Regulation and the p53 Tumor Suppressor Protein,” Critical Reviews in Eukaryotic Gene Expression, 2(3): 251-263, 1992).

The p53 gene encodes a 393-amino-acid phosphoprotein that can form complexes with host proteins such as large-T antigen and E1B. The protein is found in normal tissues and cells, but at concentrations which are minute by comparison with transformed cells or tumor tissue. Wild-type p53 appears to be important in regulating cell growth and division. Over expression of wild-type p53 has been shown in some cases to be anti-proliferative in human tumor cell lines. Thus p53 can act as a negative regulator of cell growth (Weinberg, R., “Tumor Suppressor Genes,” Science, 254:1138-1146, 1991) and may directly suppress uncontrolled cell growth or indirectly activate genes that suppress this growth. Thus, absence or inactivation of wild type p53 may contribute to transformation. However, some studies indicate that the presence of mutant p53 may be necessary for full expression of the transforming potential of the gene.

Although wild-type p53 is recognized as a centrally important growth regulator in many cell types, its genetic and biochemical traits appear to have a role as well. Mis-sense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Casey and colleagues have reported that transfection of DNA encoding wild-type p53 into two human breast cancer cell lines restores growth suppression control in such cells (Casey, et al., (1991). Growth suppression of human breast cancer cells by the introduction of a wild-type p53 gene. Oncogene 6:1791-1797). A similar effect has also been demonstrated on transfection of wild-type, but not mutant, p53 into human lung cancer cell lines (Takahashi, et al., (1992). Wild-type but not mutant p53 suppresses the growth of human lung cancer cells bearing multiple genetic lesions. 1992. Cancer Res. 52:2340-2342). The p53 appears dominant over the mutant gene and will select against proliferation when transfected into cells with the mutant gene. Normal expression of the transfected p53 does not affect the growth of cells with endogenous p53. Thus, such constructs might be taken up by normal cells without adverse effects.

It is thus possible that the treatment of p53-associated cancers with wild type p53 may reduce the number of malignant cells. However, studies such as those described above are far from achieving such a goal, not least because DNA transfection cannot be employed to introduce DNA into cancer cells within a patients' body.

2. Gene Therapy Approaches

There have been several experimental approaches to gene therapy proposed to date, but each suffers from their particular drawbacks (Mulligan, et al., (1993), Science 260:926). As mentioned above, basic transfection methods exist in which DNA containing the gene of interest is introduced into cells non-biologically, for example, by permeabilizing the cell membrane physically or chemically. Naturally, this approach is limited to cells that can be temporarily removed from the body and can tolerate the cytotoxicity of the treatment, i.e. lymphocytes. Liposomes or protein conjugates formed with certain lipids and amphophilic peptides can be used for transfection, but the efficiency of gene integration is still very low, on the order of one integration event per 1,000 to 100,000 cells, and expression of transfected genes is often limited to days in proliferating cells or weeks in non proliferating cells. DNA transfection is clearly, therefore, not a suitable method for cancer treatment.

A second approach capitalizes on the natural ability of viruses to enter cells, bringing their own genetic material with them. Retroviruses have promise as gene delivery vectors due to their ability to integrate their genes into the host genome, transferring a large amount of foreign genetic material, infecting a broad spectrum of species and cell types and of being packaged in special cell-lines. However, three major problems hamper the practical use of retrovirus vectors. First, retroviral infectivity depends on the availability of the viral receptors on the target surface. Second, retroviruses only integrate efficiently into replicating cells. And finally, retroviruses are difficult to concentrate and purify.

3. Adenovirus Constructs for use in Gene Therapy

Human adenoviruses are double-stranded DNA tumor viruses with genome sizes of approximate 36 kb (Tooza, J. (1981). Molecular biology of DNA Tumor viruses, 2nd ed. Cold Spring Harbor Laboratory, Cold Spring Harbor, N.Y.). As a model system for eukaryotic gene expression, adenoviruses have been widely studied and well characterized, which makes them an attractive system for development of adenovirus as a gene transfer system. This group of viruses is easy to grow and manipulate, and they exhibit a broad host range in vitro and in vivo. In lytically infected cells, adenoviruses are capable of shutting off host protein synthesis, directing cellular machineries to synthesize large quantities of viral proteins, and producing copious amounts of virus.

The E1 region of the genome includes E1A and E1B which encode proteins responsible for transcription regulation of the viral genome, as well as a few cellular genes. E2 expression, including E2A and E2B, allows synthesis of viral replicative functions, e.g. DNA-binding protein, DNA polymerase, and a terminal protein that primes replication. E3 gene products prevent cytolysis by cytotoxic T cells and tumor necrosis factor and appear to be important for viral propagation. Functions associated with the E proteins include DNA replication, late gene expression, and host cell shutoff. The late gene products include most of the virion capsid proteins, and these are expressed only after most of the processing of a single primary transcript from the major late promoter has occurred. The major late promoter (MLP) exhibits high efficiency during the late phase of the infection (Stratford-Perricaudet, L. and M. Perricaudet. (1991a). Gene transfer into animals: the promise of adenovirus. p. 51-61, In O. Cohen-Haguenauer and M. Boiron (Eds.), Human Gene Transfer, Editions John Libbey Eurotext, France.).

As only a small portion of the viral genome appears to be required in cis (Tooza, 1981), adenovirus-derived vectors offer excellent potential for the substitution of large DNA fragments when used in connection with cell lines such as 293 cells. Ad5-transformed human embryonic kidney cell line (Graham, et al., (1977). Characteristics of a human cell line transformed by DNA from human adenovirus type 5. J. Gen Virol. 36:59-72) has been developed to provide the essential viral proteins in trans. The inventors thus reasoned that the characteristics of adenoviruses rendered them good candidates for use in targeting cancer cells in vivo (Grunhaus, A. and Horwitx, M. S. (1992). Adenoviruses as cloning vectors. Semin. Virology 3:237-2542).

Particular advantages of an adenovirus system for delivering foreign proteins to a cell include (i) the ability to substitute relatively large pieces of viral DNA by foreign DNA; (ii) the structural stability of recombinant adenoviruses; (iii) the safety of adenoviral administration to humans; and (iv) lack of any known association of adenoviral infection with cancer or malignancies; (v) the ability to obtain high titers of the recombinant virus; and (vi) the high infectivity of Adenovirus.

Further advantages of adenovirus vectors over retroviruses include the higher levels of gene expression. Additionally, adenovirus replication is independent of host gene replication, unlike retroviral sequences. Because adenovirus transforming genes in the E1 region can be readily deleted and still provide efficient expression vectors, oncogenic risk from adenovirus vectors is thought to be negligible (Grunhaus & Horwitz, 1992).

In general, adenovirus gene transfer systems are based upon recombinant, engineered adenovirus which is rendered replication-incompetent by deletion of a portion of its genome, such as E1, and yet still retains its competency for infection. Relatively large foreign proteins can be expressed when additional deletions are made in the adenovirus genome. For example, adenoviruses deleted in both E1 and E3 regions are capable of carrying up to 10 Kb of foreign DNA and can be grown to high titers in 293 cells (Stratford-Perricaudet and Perricaudet, 1991a). Surprisingly persistent expression of transgenes following adenoviral infection has also been reported.

Adenovirus-mediated gene transfer has recently been investigated as a means of mediating gene transfer into eukaryotic cells and into whole animals. For example, in treating mice with the rare recessive genetic disorder ornithine transcarbamylase (OTC) deficiency, it was found that adenoviral constructs could be employed to supply the normal OTC enzyme. Unfortunately, the expression of normal levels of OTC was only achieved in 4 out of 17 instances (Stratford-Perricaudet et al., (1991b) Hum. Gene. Ther. 1:241-256). Therefore, the defect was only partially corrected in most of the mice and led to no physiological or phenotypic change. These types of results therefore offer little encouragement for the use of adenoviral vectors in cancer therapy.

Attempts to use adenovirus to transfer the gene for cystic fibrosis transmembrane conductance regulator (CFTR) into the pulmonary epithelium of cotton rats have also been partially successful, although it has not been possible to assess the biological activity of the transferred gene in the epithelium of the animals (Rosenfeld et al., (1992) Cell 68:143-155). Again, these studies demonstrated gene transfer and expression of the CFTR protein in lung airway cells but showed no physiologic effect.

These types of results do not demonstrate that adenovirus is able to direct the expression of sufficient protein in recombinant cells to achieve a physiologically relevant effect, and they do not, therefore, suggest a usefulness of the adenovirus system for use in connection with cancer therapy. Furthermore, prior to the present invention, it was thought that p53 could not be incorporated into a packaging cell, such as those used in the preparation of recombinant adenovirus, as it would be toxic. As E1B of adenovirus binds to p53, this was thought to be a further reason why adenovirus and p53 technology could not be combined.

4. p53-Adenovirus Constructs and Tumor Suppression

The present invention provides cancer gene therapy with a new and more effective tumor suppressor vector. This recombinant virus exploits the advantages of adenoviral vectors, such as high titer, broad target range, efficient transduction, and non-integration in target cells. In one embodiment of the invention, a replication-defective, helper-independent adenovirus is created that expresses wild type p53 (Ad5RSV-p53) under the control of the Rous Sarcoma Virus promoter. Additional embodiments of the invention include recombinant adenoviruses expressing wild type p53 under the control of other types of promoters, such as the CMV viral promoter, SV40 virus promoter, etc., as described below.

Control functions on expression vectors are often provided from viruses when expression is desired in mammalian cells. For example, commonly used promoters are derived from polyoma, adenovirus 2 and simian virus 40 (SV40). The early and late promoters of SV40 virus are particularly useful because both are obtained easily from the virus as a fragment which also contains the SV40 viral origin of replication. Smaller or larger SV40 fragments may also be used provided there is included the approximately 250 bp sequence extending from the HindIII site toward the BglI site located in the viral origin of replication. Further, it is also possible, and often desirable, to utilize promoter or control sequences normally associated with the included gene sequence, provided such control sequences are compatible with the host cell systems.

An origin of replication may be provided by construction of the vector to include an exogenous origin, such as may be derived from SV40 or other viral (e.g., polyoma, adeno, VSV, BPV) source, or may be provided by the host cell chromosomal replication mechanism. If the vector is integrated into the host cell chromosome, the latter is often sufficient.

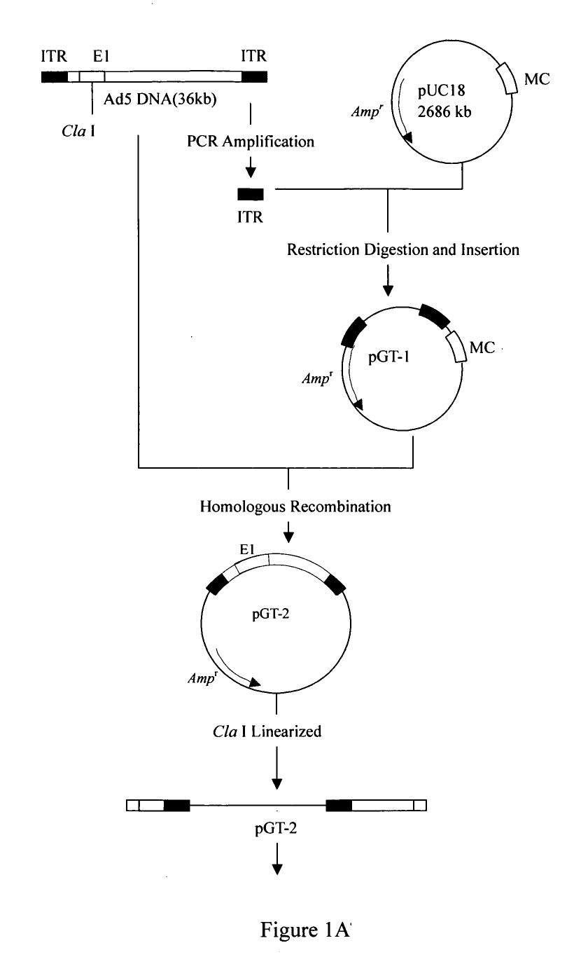

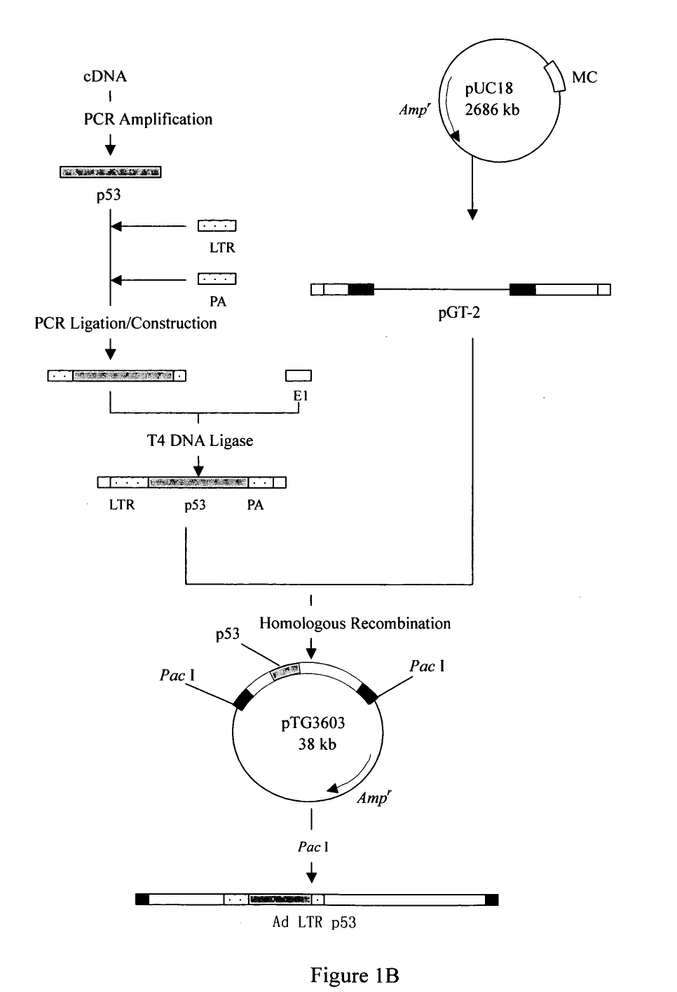

The design and propagation of the preferred p53 adenovirus is diagramed in FIG. 1. In connection with this, an improved protocol has been developed for propagating and identifying recombinant adenovirus. After identification, the p53 recombinant adenovirus was structurally confirmed by the PCR analysis. After isolation and confirmation of its structure, the p53 adenovirus was used to infect human cancer cell line 293, which has a homozygous p53 gene deletion. Western blots showed that the exogenous p53 protein was expressed at a high level.

The studies disclosed herein indicate that the p53 recombinant adenovirus possesses properties of tumor suppression, which appear to operate by restoring p53 protein function in tumor cells. These results provide support for the use of the Ad5RSV-p53 virion as a therapeutic agent for cancer treatment.

5. Improved Protocol for Propagating and Identifying Recombinant Adenovirus

Recombinant adenovirus as a new gene delivery system has many potential applications in gene therapy and vaccine development. Propagation of recombinant adenovirus is therefore an important molecular biological tool. The existing methods for propagating recombinant adenovirus use calcium phosphate precipitation-mediated transfection into 293 cells and subsequent plaque assays on the transfected cells. The transfection efficiency associated with this method needs to be improved and, also, the procedure could be simplified.

Treatment of cancer has, over the last twenty years, been the focus of a significant research and development effort. Many approaches to cancer therapy have been investigated. As a practical matter, cancer therapy can involve use of multiple treatment methods including surgical excision, radiation therapy (radiotherapy), chemotherapy, and bone marrow transplantation (for treatment in patients with some types of hematological malignancies, particularly acute myelocytic leukemia). The specific protocol utilized to treat a given malignancy, depends on the nature, location and type of malignancy being treated. Surgical excision is the preferred method for treatment of primary circumscribed tumors. Often, however, surgical excision is combined with radiation therapy and/or chemotherapy to complete the treatment protocol. In instances where the malignancy is not localized or where its location lowers the probability of successful removal or excision by surgical techniques, chemotherapy and radiation therapy are often used in combination.

Chemotherapy has been shown to produce long term remissions in patients with some types of cancer, including Hodgkin's Disease, acute lymphocytic and myelogenous leukemia, testicular cancer and non-Hodgkin's lymphoma. In other types of cancer, chemotherapy has been used successfully to decrease the size of large primary tumors prior to surgery. Chemotherapy often involves the use of combinations of chemotherapeutic agents. New protocols (programs for combination drug treatment) are being developed and tested continuously by the medical research community.

Anti-tumor agents are drugs which, in addition to killing tumor cells, can and do damage normal tissue. Even with the extensive research that has been conducted to define dosage levels and scheduling of drug administration, chemotherapy often results in unpleasant and possibly dangerous side effects due to drug toxicity. Radiation therapy produces many of the same problems. Most common of such side effects are nausea and vomiting, alopecia (hair loss), and bone marrow depression. Such side effects are usually, but not always, reversible. Some anti-cancer drugs may permanently damage the nervous system, heart, lungs, liver, kidneys, gonads or other organs. Some chemotherapeutic agents are themselves carcinogenic. Patients undergoing radiotherapy or chemotherapy must also take precautions to avoid what can be life threatening infections in their therapy-induced immuno-suppressed condition.

Treatments have been developed to counteract the side effects of cancer radiotherapy and chemotherapy. For example, drugs can be administered to provide some relief from nausea, antibiotics can be administered to help fight infection, and transfusions can be administered to increase blood cell and platelet counts if necessary.

Chemotherapeutic agents have been found useful in treating cancer in humans. Broadly classified as antineoplastics, chemotherapeutic agents found to be of assistance in the suppression of tumors include but are not limited to alkyleting agents (e.g., nitrogen mustards), antimetabolites (e.g., pyrimidine analogs), radioactive isotopes (e.g., phosphorous and iodine), hormones (e.g., estrogens and adrenocorticosteroids), miscellaneous agents (e.g., substituted ureas) and natural products (e.g., vinca alkyloids and antibiotics). Although the preceding compounds are not curative agents, they are widely recognized in the medical profession as useful in the suppression, palliation, retardation and control of malignant tumors. While these compounds have been found to be effective and are in general clinical use as antiproliferative agents, there are well recognized drawbacks associated with their administration. The alkylating agents have marked cytotoxic action and the ability of these drugs to interfere with normal mitosis and cell division can be lethal. The antimetabolities can lead to anorexia, progressive weight loss, depression, and coma. Prolonged administration of antimetabolites can result in serious changes in bone marrow. Both the alkylating agents and the antimetabolities generally have a depressive effect on the immunosuppressive system. Prolonged administration of natural products such as vinca alkyloids can also result in bone marrow depression. Hydroxy urea and other chemically derived agents can lead to rapid reduction in levels of adrenocorticosteroids and their metabolites. The administration of hormonal compounds or radioactive isotopes is also undesirable from the viewpoint of inflicting damage on the immunosuppressive system and thereby disabling the body's defenses against common infections. In most instances, it would be preferable to employ a chemotherapeutic agent which is effective in controlling, retarding, or suppressing the growth of malignant tumors while simultaneously acting to stimulate the patient's immune system.

Chemotherapy treatment is given either in a single or in several large doses or, more commonly, it is given in small doses 1 to 4 times a day over variable times of weeks to months. There are many cytotoxic agents used to treat cancer, and their mechanisms of action are generally poorly understood.

Irrespective of the mechanism, useful chemotherapeutic agents are known to injure and kill cells of both tumors and normal tissues. The successful use of chemotherapeutic agents to treat cancer depends upon the differential killing effect of the agent on cancer cells compared to its side effects on critical normal tissues. Among these effects are the killing of hematopoietic blood forming cells, and the killing and suppression of the white blood cells, which can lead to infection. Acute and chronic bone marrow toxicities are also major limiting factors in the treatment of cancer. They are both related to a decrease in the number of hemopoietic cells (e.g., pluripotent stem cells and other progenitor cells) caused by both a lethal effect of cytotoxic agents or radiation on these cells, and via differentiation of stem cells provoked by a feed-back mechanism induced by the depletion of more mature marrow compartments. (U.S. Pat. No. 5,595,973 incorporated by reference herein in its entirety.) Stimulators and inhibitors of bone marrow kinetics play a prominent role in the induction of damage and recovery patterns (Tubiana, et al., (1993) “Ways of Minimising Hematopoietic Damage Induced by Radiation and Cytostatic Drugs—The Possible Role of Inhibitors,” Radiothereapy and Oncology, 29: 1-17).

Prevention of, or protection from, the side effects of chemotherapy would generate great benefit to cancer patients. There have been many previous efforts to reduce these side effects and they were largely unsuccessful. For life-threatening side effects, efforts have concentrated on altering the dose and schedules of the chemotherapeutic agent to reduce the side effects. Other options are becoming available, such as the use of granulocyte colony stimulating factor (G-CSF), granulocyte-macrophage-CSF (GM-CSF), epidermal growth factor (EGF), interleukin 11, erythropoietin, thrombopoietin, megakaryocyte development and growth factor, pixykines, stem cell factor, FLT-ligand, as well as interleukins 1, 3, 6, and 7, to increase the number of normal cells in various tissues before the start of chemotherapy (See Joaquin J. Jimenez and Adel A. Yunis, “Protection from 1-.beta.-D-Arabinofuranosylcytosine-Induced Alopecia by Epidermal Growth factor and Fibroblast Growth Factor in the Rat Model,” cancer Research, vol. 52 (1992) pp. 413-415). The mechanisms of protection by these factors, while not fully understood, are most likely associated with an increase in the number of normal critical target cells before treatment with cytotoxic agents, and not with increased survival of cells following chemotherapy.

As an example of side effect of cancer therapy, acute myelosuppression often occurs as a consequence of cytotoxic chemotherapy and is well recognized as a dose-limiting factor in cancer treatment. (U.S. Pat. No. 5,595,973). Although other normal tissues may be adversely affected, bone marrow is particularly sensitive to the proliferation-specific treatment such as chemotherapy or radiotherapy. For some cancer patients, hematopoietic toxicity frequently limits the opportunity for chemotherapy dose escalation. Repeated or high dose cycles of chemotherapy may be responsible for severe stem cell depletion leading to serious long-term hematopoietic sequelea and marrow exhaustion.

The methods disclosed in the present invention are effective in reducing the side effects caused by, as an example, chemotherapy, using any cytotoxic chemotherapy agent, including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al., Cancer Chemotherapeutic Agents: Handbook of Clinical Data (1978), hereby incorporated by reference.

The methods of the invention are also particularly suitable for those patients in need of repeated or high doses of chemotherapy or radiotherapy. For some cancer patients, toxicity frequently limits the opportunity for chemotherapy dose escalation. Repeated or high dose cycles of chemotherapy may be responsible for severe stem cell depletion leading to severe long-term hematopoietic sequelea and marrow exhaustion. The methods of the present invention provide for improved mortality and blood cell count when used in conjunction with chemotherapy.

The present invention discloses the use of compositions, also know as “pharmaceutical composition” in reducing the side effects of various types of cancer therapy, and improving the quality of life for cancer patients. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients such as the recombinant p53 adenovirus, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol; and sorbitol; starch from corn, wheat, rice, potato, or other plants; cellulose, such as methyl cellulose, hydroxypropylmethylcellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

A therapeutically effective dose refers to that amount of active ingredient, for example a recombinant p53 adenovirus, which ameliorates the symptoms or conditions of the side effects of cancer treatment for the patient, and improve the quality of life in the patient's lives. Therapeutic efficacy and toxicity may be determined by standard pharmaceutical procedures in cell cultures or with experimental animals, such as by calculating the ED₅₀ (the dose therapeutically effective in 50% of the population) or LD ₅₀ (the dose lethal to 50% of the population) statistics. The dose ratio of therapeutic to toxic effects is the therapeutic index, and it can be expressed as the ED₅₀/LD₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner with ordinary skills in the art, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 g to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

The present invention discloses the usage of a gene therapy product such as recombinant p53 adenovirus in combination with radiotherapy, using radiation and waves that induce DNA damage, such as, gamma-irradiation, X-rays, UV-irradiation, microwaves, electronic emissions, and the like. The above-mentioned agents, together with chemotherapy agents can be classified as DNA damaging agents. A variety of chemical compounds, also described as “chemotherapeutic agents”, function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP), and even hydrogen peroxide. The invention also encompasses the use of a combination of one or more DNA damaging agents, whether radiation-based or actual compounds, such as the use of X-rays with cisplatin or the use of cisplatin with etoposide.

### EXAMPLES

The following examples are provided to illustrate but not limit the invention. The present invention may be better understood with reference to the accompanying example that is intended for purposes of illustration only and should not be construed to limit the scope of the invention, as defined by the claims appended hereto.

### Example One

### Preparation Of Gendicine, A Recombinant P53 Adenovirus

A recombinant p53 adenovirus was prepared and used for testing in the clinical trials. The recombinant adenovirus is named “Gendicine”. The detailed process describing the preparation of Gendicine is disclosed in Chinese Patent No.: ZL 02115228.4, the entire content of which is incorporated by reference herein.

The process can be described as follows. Recombinant viral vector was generated through homologous recombination in prokaryotic cells. First, we made recombinant pGT-2, using adenovirus and pGT-1, through homologous recombination in E. coli. Next, we made a construct consisting of adenovirus Right Side Arm promoter-p53 cDNA-Poly A tail/Adenovirus Left Side Arm, and combined this construct with pGT-2 to produce the recombinant pGT-3. Restriction digestion of pGT-3 using PacI enabled us to eliminate prokaryotic plasmid sequences, after which we were able to produce the purified recombinant p53 adenovirus.

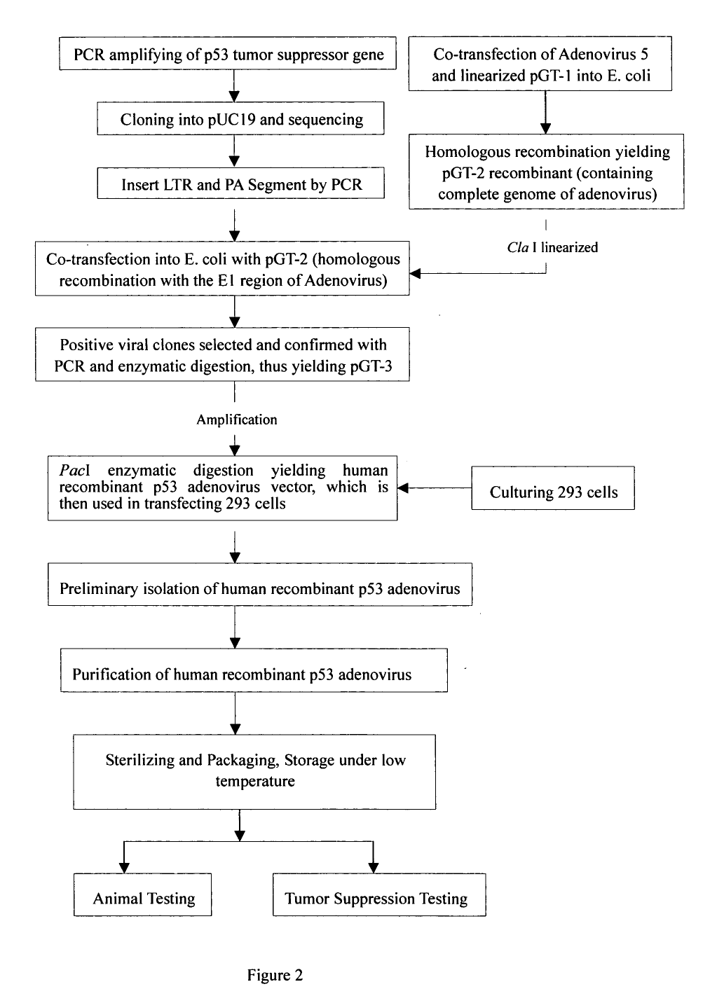

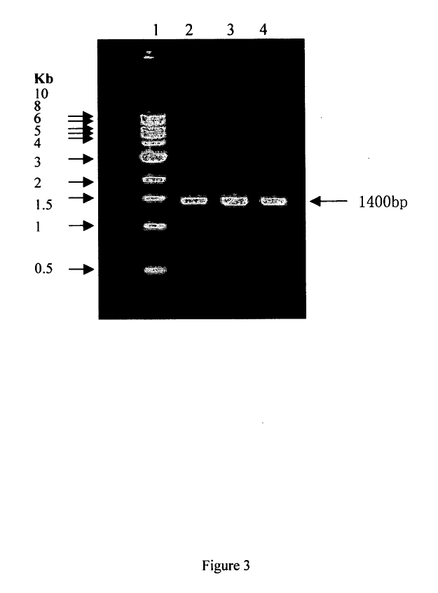

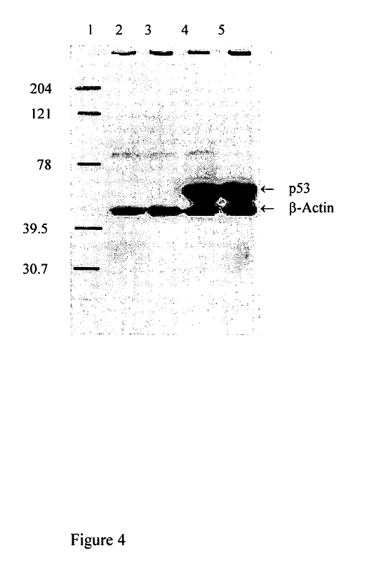

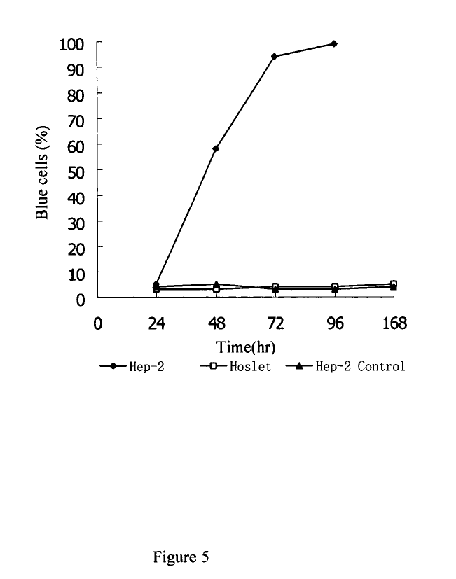

As illustrated in FIG. 1 and FIG. 2, the construction and production of the human recombinant p53 adenovirus Gendicine can be described as follows. First, adenovirus vector and shuttle vector containing p53 gene were introduced together into E. coli so as to generate recombinant adenovirus containing p53 gene, and such recombinant adenovirus was selected from positive recombinant clones. The recombinant clones were amplified in 293 cells, and through multiple steps of purification, clinical grade human recombinant p53 adenovirus was harvested. FIG. 3 shows the results of determination of the structural stability of the human p53 adenovirus. The result in FIG. 3 shows that after many generations, the recombinant adenovirus maintained its structural stability. FIG. 4 shows that the Western Blot results indicating that human recombinant p53 adenovirus can be highly expressed in throat cancer cells Hep-2, and non-small lung cancer cells H1299. FIG. 5 is a graph showing that the recombinant p53 adenovirus has killing effect on Hep-2 cells. The killed cells are represented by the color blue as stained with Trypan Blue.

### Example Two

### Clinical Trials of Head and Neck Squamous Cancer Patients

Recombinant adenovirus p53 anticancer injection (Gendicine) was provided by Shenzhen Sibiono Gene Tech Co. in Shenzhen, China, Batch No. was #S010731, and the medication was packaged in 1×10¹² VP/ml/Each dose, and each dose was contained in a 2 ml size Saline Bottle. There was one bottle in each box.

The clinical trial was designed as multi-center, concurrent control, single-agent, open label, randomized clinical trial. The selection process for patients was as follows: First, the patient signed consent form to participate in clinical trials. Using pathological testing methods, it was confirmed that the patients indeed had head and neck squamous cell carcinoma patients. Their cancer is then classified according to the UICC TNM Classification method. The patients selected into the trial possessed measurable cancer masses that were readily observable, and accessible for local injection of drugs. The patient ages ranged from 18 years old to 80 years old, and their life expectancy was at least 3 months. Both male and female patients were included.

Exclusion criteria were chosen as follows: Patients suffering from acute upper respiratory infection, or those having uncontrolled fever derived from such infection were excluded. Patients were also excluded if the cancer masses were being treated with other locally-administered medication. Those patients with serious heart, liver, lung, or kidney diseases, patients who tended to bleed easily, patients who were pregnant women or nursing mothers were also excluded.

During the trials, patients were eliminated if they met the following standards: patients who could not bear the high fever caused by using the recombinant medicine; patients who much stop the trial because of the onset of unexpected toxic side effects; and those patients who withdrew from the trials for personal reasons. These clinical trial lasted from June 2001 to May 2003, altogether with 155 cases in four hospitals.

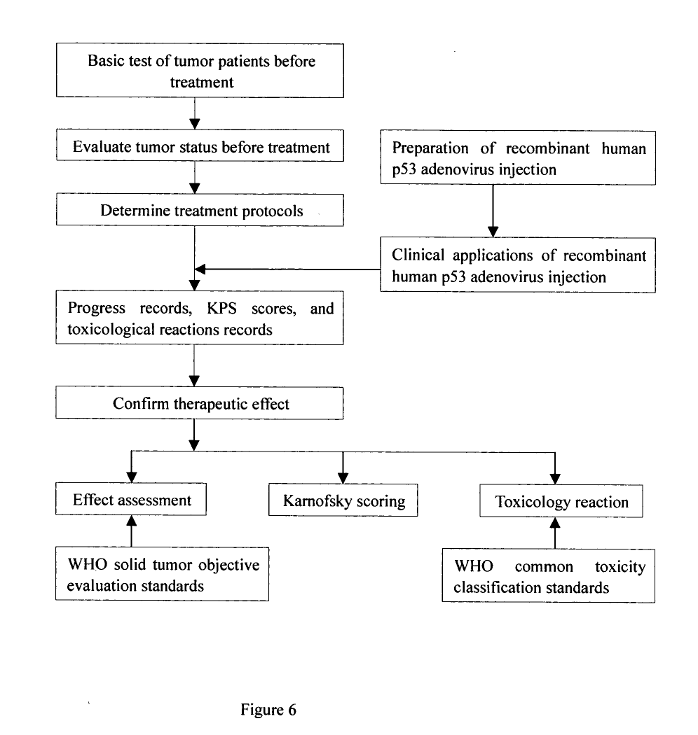

The clinical trial protocol was designed as shown in FIG. 6, and can be described as follows:

1. Group of Gene Therapy (GT) Combined with Radiotherapy (RT)

Each Friday, Gendicine was injected inside of the tumor, at a dose of 10¹² VP/each injection. Three days after the injection, radiotherapy is performed on the patient. The radiation is done using standard or intensity modulated 3-dimensional conformal radiation therapy method, dosage was 70Gy/35f for about 7-8 weeks. After five weeks, and cumulative dosage of 40Gy, the first cycle is completed. After eight weeks of cumulative dosage of 70Gy, the second cycle is completed. At the 12^{th} week, the therapeutic results are confirmed using CT scanning. Using the WHO solid tumor objective evaluation standards, we calculated the cancer reduction rate (percentage) at points corresponding to 40Gy, 70Gy, and the 12^{th} week. The therapeutic results are classified as Complete Response (CR), Partial Response (PR), Stable Disease (SD), and Progressive Disease (PD).

Here, 70Gy refers to total radiation dosage. 35f refers to the total number of days of radiation. 7-8 weeks refers to the length of treatment cycle. For example, patients undergo radiation for five days out of each week, with two days for resting. On each day of radiation, the average radiation dosage is 2Gy per day.

2. Group of Gene Therapy (GT) Group Combined with Chemotherapy (CT)

Tumor solid mass is injected with Gendicine one dosage per week. The whole treatment period is 8 weeks, and 8 injections are performed. Chemotherapy starts three days after Gendicine injection. DDP (Cis-Platinum) dosage was 80 mg/m², using intravenous injection on day one. From Day one to Day Five, 5-Fluororacil was continuously injected using intravenous injection at a dosage of 500 mg/m² .

3. Clinical Lab Testing Methods:

Regular testing of the patients begins within one week after patient is admitted into the clinical trial, and weekly after the start of the clinical trial. The testing items included physical examination, KPS (Karnofsky Performance Status) scoring, complete blood test, complete urine test, and complete stool test. Every month, biochemical tests are performed, including Blood Urea Nitrogen (BUN), Cr (Creatinine) test, Aspartate Transaminase (AST) and Alanine Transaminase (ALT). EKG and chest X-ray are also examined. Each week, we used the WHO classification standards for evaluating acute and sub-acute toxicity for anticancer drugs in determining the toxicity of Gendicine in these trials. The categories are light (I), medium (II), serious (III), and Life-critical (IV). These categories were determined and recorded. According to the testing results of the Phase I clinical results, changes in body temperature was especially noted.

4. Results Analysis, and Statistical Calculations

All statistical analysis was performed using the SPSS11.0 statistics software. The testing was done according to ITT testing method. Comparison of the Reduction of the size of the cancer mass was done using t test. Comparison of the therapeutic significance was done using the Pearson Chi-Square test.

5. Effect on Blood, Urine, Stool, and Effect on Liver and Kidney Function.

TABLE 1
GT/RT Testing Group with 37 cases of patients using their
complete blood count and blood biochemistry testing.

| | No Drug | | 8 weeks | P | Value |
|---|---|---|---|---|---|
| Items | (A) | 4 weeks(B) | (C) | (A-B) | (A-C) |
| WBC (10⁹/L) | 6.81 ± 2.10 | 5.48 ± 1.47 | 5.41 ± 1.58 | 0.003^{▴} | 0.002^{▴} |
| Hemoglobin | 134.05 ± 15.08 | 126.03 ± 14.73 | 127.28 ± 14.51 | 0.040^{▴} | 0.095 |
| (g/L) | | | | | |
| Platelet (10⁹/L) | 250.49 ± 69.67 | 242.19 ± 86.74 | 250.58 ± 110.33 | 0.893 | 1.000 |
| AST (U/L) | 27.59 ± 13.70 | 25.51 ± 12.82 | 26.08 ± 11.16 | 0.701 | 0.829 |
| ALT (U/L) | 31.14 ± 29.39 | 27.84 ± 28.44 | 26.76 ± 27.06 | 0.835 | 0.734 |
| BUN (μmol/L) | 5.51 ± 1.12 | 4.83 ± 1.21 | 4.80 ± 1.66 | 0.046^{▴} | 0.110 |
| Cr (μmol/L) | 80.59 ± 12.37 | 78.64 ± 11.21 | 77.53 ± 11.41 | 0.698 | 0.429 |

A delta symbol (^{▴}) shows that there are differences between the two compared groups (P < 0.05)

The results in Table 1 indicate that, as compared between pre-injection of Gendicine, and two weeks after Gendicine Injection, Complete Blood Count and Blood Biochemistry testing were all within normal range. Before Injection of Gendicine, ALT, AST, BUN and Cr value were normal, and after two weeks of Gendicine injection, their values were only slightly reduced. Chest X-ray and EKG showed no changes before and after Gendicine injection.

### Example Three

### Gendicine Therapy Combined with Chemotherapy of Liver Cancer Patients

Human recombinant p53 adenovirus, Gendicine, was prepared as described in Example One. In this example we describe a clinical trial showing the beneficial effects of Gendicine on liver cancer patients who have undergone chemotherapy.

This clinical trial was designed as single-center, concurrent control, single-agent, open label, randomized clinical trial. The patient selection standards were as follows: First, they signed consent agreement to participate in the clinical trial. Pathological and histological tests confirmed that they carried Hepatocellular carcinoma (HCC). The evaluation standards for HCC satisfied the Chinese Standard Protocols for the Diagnosis and Treatment of Common Malignancies. The tumor must possess measurable cancer mass that were easily observed and can be injected using local drug injection. They ages belonged in the range of 18-75, with life expectancy of at least 3 months. Both male and female patients were eligible.

The exclusion criteria were the same as in Example One, which were as follows: Patients suffering acute upper respiratory infection, or those whose having uncontrolled fever derived from such infection are excluded. Patients are also excluded if the cancer masses are being treated with other medication locally administered. Those patients with serious heart, liver, lung, or kidney diseases, patients who easily bleed, patients who are pregnant women or nursing mothers are also excluded.

Similarly as in Example One, during the trials, patients were eliminated if they meet the following standards: patients who cannot take the high fever caused by using the recombinant medicine; patients who much stop the trial because of the onset of unexpected toxic side effects; and those patients who withdrew from the trials for personal reasons.

From March 2004 to July 2004, there were 35 cases in the liver cancer clinical trial of Gendicine Gene Therapy and chemotherapy combination therapy.

1). Therapeutic Protocols:

There were altogether 75 HCC patients in this clinical trial, with 51 males, and 24 females. The HCC cancer patients were at mid to late stages.

Two groups were randomly established. The first group of 40 patients went through simple chemotherapy, using trans-catheter hepatic arterial chemo-embolization (TACE). Using routine femoral artery puncture, the catheter was implanted into celiac artery, or common hepatic artery. Afterwards, we used superior mesenteric arterial imaging to determine tumor number, location, type, size, supply blood vessels, and arteriovenous fistulas. To identify liver cancer, and other supplying arteries, we used gelatin sponge embolism to inject into the hepatic artery 5-fluoracil, Adriamycin (ADM), Mitomycin (MMC), DDP and Hydroxycamptothecin (HCPT), in order to execute chemotherapy. After that, we introduced catheter super-selectively into the blood supplying branches of the hepatic artery, and inject the mixture of 10 mg ADM and 10-30 ml Iodinate Oil into the liver slowly under the monitor of X-ray. TACE was injected every 4 weeks for total of two injections, when the therapeutic effects were evaluated.

The second group of 35 patients went through the GT and chemotherapy combination therapy. 48-72 hours before the TACE chemotherapy, Gendicine was injected under the guidance of CT using percutaneous intratumor injection at multiple spots in the tumor. Based upon the size of the tumor, the dosage was about 1-4×10¹² VP each time, once a week, 3-4 injections continuously. The TACE chemotherapy was administered as described above.

Using the WHO solid tumor objective evaluation standards, the therapeutic results are classified as Complete Response (CR), Partial Response (PR), Stable Disease (SD), and Progressive Disease (PD).

2). Standard Clinical Testing Results.

Regular testing of the patients begins within one week after patient is admitted into the clinical trial, and weekly after the start of the clinical trial. The testing items included physical examination, KPS (Karnofsky Performance Status) scoring, complete blood test, complete urine test, and complete stool test. Every month, biochemical tests are performed, including Blood Urea Nitrogen (BUN), Cr (Creatinine) test, Aspartate Transaminase (AST) and Alanine Transaminase (ALT). EKG and chest X-ray are also examined. Each week, we used the WHO classification standards for evaluating acute and sub-acute toxicity for anticancer drugs in determining the toxicity of Gendicine in these trials. The categories are light (I), medium (II), serious (III), and Life-critical (IV). These categories were determined and recorded. According to the testing results of the Phase I clinical results, changes in body temperature was especially noted. Regular evaluation indicators are scored using the KPS methodology.

3). Results Analysis and Statistical Results.

All data were analyzed using the SPSS11.0 statistical software.

4). Effect on White Blood Cells Count, Symptoms, and KPS Scores.

See Table 2 for the effect on white blood cells. It can be seen that for the control group of simple TACE chemotherapy, the white blood cell count is decreased significant. Comparing to the test group of GT and Chemotherapy combination therapy, the difference was significant, with p<0.05.

TABLE 2
Reduction in White Blood Cell Count
(×10⁹/L) and Number of Cases (%).

| | | | | Total Number of |
|---|---|---|---|---|
| | Degree of Reduction of WBC | | | Cases showing |
| Groups | 4.0-3.0 | 3.0-2.0 | 2.0 or lower | Reduction |
| GT-TACE | 12 (25.0) | 4 (8.3) | 2 (4.2) | 18 (37.5) |
| TACE | 8 (13.3) | 20 (33.3) | 11 (18.3) | 39 (65.0) |

Tables 3 shows the results in the improvement of the well-being of the patients. The numbers inside of the parentheses are percentages. It can be from Table 3 that, after one month of treatment, the GT-TACE group (Gendicine combined with Chemotherapy) showed drastic improvement in disease symptoms. Comparing to the control group of simple TACE Chemotherapy, the difference was significant, with p<0.05.

TABLE 3
Improvement of patients who were treated
with GT-Chemo combination therapy.

| | Number | | Digestive | |
|---|---|---|---|---|
| Groups | of Cases | Fever | Reaction | Reduced Pain |
| GT-TACE | 35 | 25 (71.4) | 16 (45.6) | 30 (85.7) |
| TACE | 40 | 18 (45.0) | 24 (60.0) | 21 (52.5) |

Table 4 shows the improvement of the combination therapy cancer patients in terms of their KPS scoring. It can be seen from Table 4 that, after one month of treatment, the GT-TACE group of patients shows significant improvement in terms of its KPS scoring.

TABLE 4
KPS Scoring changes after one month of combination therapy

| | | Increase | Increase | No | | Total of |
|---|---|---|---|---|---|---|
| Group | Cases | 20 | 10 | change | Reduced | Increased |
| GT- | 35 | 7 | 15 | 9 | 4 | 22 (53.3) |
| TACE | | | | | | |
| TACE | 40 | 9 | 10 | 8 | 13 | 19 (47.5) |

The results in this Example demonstrate that a combination of Gene therapy and chemotherapy can generate significant improvements in reducing the tumor size of patients, enhancing the symptoms and well-being of the patient in a clinical setting, as compared to the control group of using chemotherapy alone. For example, the white blood count, KPS scoring an all higher than those in the control group of using only a simple TACE therapy. In addition, the appetite of the patients and the sleeping condition improved a great deal. This indicates that the human recombinant p53 adenovirus product can alleviate the side effects caused by chemotherapy. It can also improve the well-being of cancer patients both physically, and mentally.

### Example Four

### Additional Individual Success Cases Demonstrating the Efficacy of Recombinant p53 Adenovirus in Improving the Quality of Life of Cancer Patients

We demonstrate here that Gendicine was also capable of improving the quality of life in cancer patients without concurrent or sequential combination with other cancer therapy.

A 40-year old woman suffering from an invasive left breast cancer had previously undergone various forms of cancer therapy. Surgery was also performed after the discovery of the cancer, including masectomy and auxiliary lymphnode glandular excision. After the surgery, it was discovered that, out of 12 lymph nodes that were removed surgically, ten had cancer cell metastases. Later on, combination of radiotherapy at the dose level of 45Gy/25f/5w, and 9 cycles of chemotherapy of Ciclofosfamid plus Erubicin plus 5-fluoracil was performed on the patient. However, six month after the combination therapy, metastasized tumor was multiple spine regions including cervical vertebra, thoracic vertebra, and lumbar vertebra. Additional treatment of chemotherapy with Taxotere, Vinoralbine, and the newly found oral chemotherapy drug Xeloda, was performed on the patient. During the intervening periods of chemotherapy, the patient used Zometa to maintain bone stability. But, all of the above methods failed to produce the desired therapeutic effect. By January 2004, the patient showed increased level of ALT and AST, and blood count. Her physical condition started to deteriorate, and she started to feel body aches, fatigue, weight loss, mental depression, etc, indicating her cancer had worsened.

After failing to achieve effective chemotherapy and radiotherapy in her local hospital, the patient undertook one treatment of Gendicine therapy in Guang Zhou Sun Yat-Sen University Cancer Hospital. The patient then brought home Gendicine to continue the therapy, and has reported back her progress in the therapy.

Over the course of six months of administering of Gendicine, the patient felt a fantastic change in her physical well-being, including higher energy, good appetite. After the initial injection, patient's body temperature reached up to 40 degrees Celsius, with nausea, and vomiting. The initial negative reaction quickly subsided after a few days. Then, the patient became much more energetic, and was pain free, and was able to perform her daily routine in work and life.

In another case, a 60-year old woman was diagnosed with an aggressive 5 cm cancerous tumor in her pancreas, and was given one year to live. She decided not to go with the routine chemotherapy due it its associated side effects. Instead, she sought out Gendicine treatment and underwent 20 treatments in 8 weeks using intravenous injection of Gendicine.

After the treatment of Gendicine, CT Scan confirmed that the tumor in her pancreas had not grown and small lesions on her liver had disappeared. After further observation, it was found that the tumor became stable, the suspicious lesions were gone, and the enlarged lymph nodes had shrunk.

In summary, we have found that recombinant p53 adenovirus has a surprisingly unique property when it is injected into cancer patients. That is, when combined with CT or RT, it can alleviate the side effects of the RT or CT which exerted on cancer patients when they go through CT or RT alone. This discovery is significant because after the injection of Gendicine, cancer patients, especially late stage cancer patients, their physical well-being have significantly improved. For example, their mental well-being and spirits became high, their appetite got better. We postulate that this effect is due to Gendicine's global regulation of the nerve-endocrine-immune system. Thus, Gendicine is able to improve and strengthen the function of various organs of the patients, and improve their health overall.

The mechanism of action may be attributed to the structure and function of Gendicine. Gendicine is a viral particle manufactured by genetic engineering. It can stimulate the body's nervous system, endocrine system, and immune system. Gendicine is able to modulate these three systems so they can produce a series of nerve factors, hormones, and cellular factors. As seen from the examples above, one phenomenon experienced by patients after administration of Gendicine is increased body temperature, in other words, fever. This phenomenon can be explained by the increased activity of the patient body's immune system. In this case, the addition of Gendicine into the patient's body stimulated the immune response of the body. It appears that through the global regulation of the nerve-endocrine-immune network, the patient's immune capability is increased, thus it can effectively increase the killing of cancer cells by NK cells and CTL cells. Gendicine can also act to regulate physiological function of the patient, and therefore improve the patient's overall health.

Although the foregoing invention has been described in some detail by way of illustration and example for purposes of clarity of understanding it will be apparent to those skilled in the art that certain changes and modifications will be practiced. Therefore, the description and examples should not be construed as limiting the scope of the invention, which is delineated by the appended claims. In addition, all patents, patent applications, literature, papers, protocols, and other written materials mentioned and referred herein are incorporated by reference in their entirety.

1. A method for reducing side effects caused by a cancer therapy in a subject receiving said cancer therapy, comprising administering a composition comprising a recombinant p53 adenovirus and a pharmaceutically acceptable carrier to said subject in an amount effective to reduce said side effects.

2. The method of claim 1, wherein said composition is administered prior to the cancer therapy.

3. The method of claim 1, wherein said composition is administered simultaneously with the cancer therapy.

4. The method of claim 1, wherein said composition is administered subsequent to the cancer therapy.

5. The method of claim 1, wherein said cancer therapy can be either chemotherapy or radiotherapy.

6. The method of claim 1, wherein said composition is administered subsequent to a chemotherapy agent.

7. The method of claim 1, wherein said composition is administered subsequent to radiotherapy.

8. The method of claim 1, wherein said composition is administered on each of a plurality of days prior to a chemotherapy agent.

9. The method of claim 1, wherein said composition is administered on each of a plurality of days prior to a radiotherapy agent.

10. The method of claim 1, wherein said composition is administered simultaneously with a chemotherapy agent.

11. The method of claim 1, wherein said composition is administered simultaneously with a radiotherapy agent.

12. The method of claim 1, wherein a single administration of the composition is administered one day immediately prior to administration of a chemotherapy agent.

13. The method of claim 1, wherein a single administration of the composition is administered on each of two days immediately prior to administration of said chemotherapy agent.

14. The method of claim 1, wherein a single administration of the composition is administered on each of three days immediately prior to administration of said chemotherapy agent.

15. The method of claim 1, wherein said subject is a mammal.

16. The method of claim 1, wherein said subject is a human.

17. A method of improving the quality of life of a cancer patient, comprising administering a composition comprising a recombinant p53 adenovirus and a pharmaceutically acceptable carrier to said cancer patient in an amount effective to improve the quality of life of said cancer patient.

18. A method of improving the quality of life of a cancer patient, comprising
a). administering a composition comprising a recombinant p53 adenovirus and a pharmaceutically acceptable carrier to said patient in an amount effective to improve the quality of life of said cancer patient; and
b). obtaining an indication of improved quality of life for said cancer patient, said indication comprising better appetite, weight gain, higher lever of energy, less pain, and better sleep.
